(19) United States
(12) Patent Application Publication
(10) Pub. No.: US 2025/0058116 A1
(43) Pub. Date: Feb. 20, 2025

(54) PHOTOBIOMODULATION OF BASAL FOREBRAIN TO IMPROVE COGNITION

(71) Applicant: Boston Scientific Neuromodulation Corporation, Valencia, CA (US)

(72) Inventors: Jianwen Gu, Richmond, TX (US); Changfang Zhu, Valencia, CA (US)

(73) Assignee: Boston Scientific Neuromodulation Corporation, Valencia, CA (US)

(21) Appl. No.: 18/804,667

(22) Filed: Aug. 14, 2024

Related U.S. Application Data
(60) Provisional application No. 63/533,045, filed on Aug. 16, 2023.

(51) Int. Cl. A61N 1/36; A61N 5/06

(52) U.S. Cl. CPC A61N 1/36082; A61N 1/36171; A61N 5/0618; A61N 5/0622; A61N 2005/0659; A61N 2005/0663

(57) ABSTRACT

A multi-modal method of stimulation to the basal forebrain to enhance the neuroprotective effects of deep brain stimulation and photobiomodulation. Stimulation is generated by a lead that incorporates optical devices and electrodes placed either intracranially or intranasally. By using this combination therapy, both electrical and optical stimulation, and its therapeutic benefits, can be incorporated within one treatment regimen.

### CROSS REFERENCE TO RELATED APPLICATIONS

The present application claims the benefit of U.S. Provisional Patent Application No. 63/533,045, filed Aug. 16, 2023, titled PHOTOBIOMODULATION OF BASAL FOREBRAIN TO IMPROVE COGNITION, the disclosure of which is incorporated herein by reference.

### BACKGROUND

In the treatment of degenerative cognitive diseases, a promising target is the nucleus basalis of Meynert (“NBM”) within the basal forebrain. The NBM of the patient is chosen because of its large population of cholinergic neurons, which play a fundamental role in the cognitive processes related to memory. Degeneration of the NBM is accompanied by a decrease in cholinergic neurotransmission, giving rise to cognitive impairment. As the cholinergic neurons in the basal forebrain degenerate, so does the cortical cholinergic input from the NBM, predicting memory loss. New and/or alternative treatments and systems for providing treatments to the NBM and/or other brain structures to arrest, delay, or prevent degeneration, as well as to address symptoms of disease affecting the NBM are desired.

### OVERVIEW

The present inventors have recognized, among other things, that a problem to be solved is the need for new and/or alternative methods and systems for providing a neuroprotective effect on cognitive diseases, such as Alzheimer's Disease and Parkinson's disease dementia, that may lead to memory loss.

The present invention relates generally to a multi-modal method of bringing about the neuromodulation effects primarily from deep brain stimulation (DBS) and neuroprotective effects primarily from photo-bio-modulation (PBM) of the basal forebrain. In pre-clinical testing, PBM has been preliminarily shown to have potential neuroprotective effects, improve cognition in patients with dementia, and mitigate Alzheimer's disease pathology. When applied to the basal forebrain PBM may prevent or slow degeneration of the basal forebrain related to neurological or cognitive diseases. Inducing PBM via optical stimulation may result in an increased blood flow, reduced oxidative stress, and improved neural network activity and may improve tasks or functions associated with the stimulated region or subsector. Another neuromodulation technique to bring about a protective effect against, or to reduce symptoms caused by neurological disorders and cognitive diseases, is deep brain stimulation (“DBS”). DBS operates by electrically stimulating selected regions of brain tissue. Currently, this surgical procedure is used to treat or manage neurological disorders like Parkinson's disease, dystonia, and epilepsy. Electrically stimulating the targeted region or subsector can also protect and preserve the health and function of cholinergic neurons, thus mitigating the progression of neurodegenerative diseases.

Although DBS has provided significant improvement for managing motor symptoms of neurological diseases like Parkinson's, it has not been shown to provide a significant therapeutic effect on psychological and cognitive symptoms, such as Parkinson's disease dementia. DBS of the NBM has potential for patients with mild or moderate cognitive diseases, but for advanced cases, the therapeutic effects of NBM-DBS on neuropsychiatric symptoms, such as PDD, are significantly lower, if present at all. For example, DBS of the NBM has been found to have some positive effect on short-term memory, but not so for long-term memory.

PBM has been evaluated in several studies for its cognitive enhancement effects. Significant improvements in cognitive function were observed in healthy volunteers in executive test, memory test and mental flexibility. Current methods of PBM can easily allow transcutaneous stimulation, making it popular among physical therapists for sports injuries, however application to the brain presents more difficulties because the skull provides a significant barrier to light delivery.

In some examples, PBM can be provided along with DBS from a single device or separate devices. Further, in some examples of the present invention, the DBS lead is modified to allow for the placement of light-emitting devices, makes the application of PBM to the relevant brain tissue more achievable. In other examples, a device can be placed in a sinus cavity of the patient to bring a light emitting device into proximity with the NBM, enabling PBM to avoid the difficulty of passing through the skull. PBM via a sinus cavity can be provided along with DBS from a standard DBS lead, or may be used in conjunction with electrical stimulus from the sinus cavity.

In the present invention, the different energy modalities stimulate the same target to enhance the neuroprotective effects of both modalities. By combining these modalities, the therapeutic benefits and preventative benefits of optical and electrical stimulation can be incorporated within one treatment regimen.

A first illustrative and non-limiting example takes the form of a system for treating a patient comprising an implantable pulse generator containing operational circuitry adapted to control the implantable pulse generator and including generation circuitry for controlling delivery of electrical signals and optical signals; a lead having a proximal end for coupling to the implantable pulse generator and a distal end for placement in a target region of a patient's brain, the distal end including a plurality of electrodes and at least one optical device, wherein the operational circuitry is configured to control the generation circuitry to perform the following: optically modulating a target of at least one subsector or anatomic region of the basal forebrain by photobiomodulation (PBM) generated from the optical device while the distal end of the lead is located at, adjacent to, or near the basal forebrain; and electrically modulating a portion of the basal forebrain with an electrical signal generated from a first electrode positioned on the lead.

Additionally or alternatively, the operational circuitry is configured to issue the optically modulating and electrically modulating signals such that the combination of optical and electrical modulating prevents the degeneration of a patient's Nucleus Basalis of Meynert. Additionally or alternatively, the control circuitry is configured so that the first electrode is selected such that the target and the portion are spatially overlapping, and the control circuitry controls the electrically modulating and optically modulating steps to occur concurrently.

Additionally or alternatively, the control circuitry is configured so that the first electrode is selected such that the target and the portion are spatially overlapping, and the control circuitry controls the electrically modulating and optically modulating steps to occur in an alternating or interleaving fashion. Additionally or alternatively, the control circuitry controls the electrically modulating and optically modulating steps so that the interleaving fashion includes a first period in which the target is modulated, and a second period in which the portion is modulated, and the first period overlaps a portion of the second period. Additionally or alternatively, the control circuitry controls the electrically modulating and optically modulating steps so that the interleaving fashion includes a first period in which the target is modulated, and a second period in which the portion is modulated, and the first period des not overlap a portion of the second period.

Additionally or alternatively, the control circuitry is configured so that the first electrode is selected such that the target and the portion are not spatially overlapping, and the electrically modulating and optically modulating steps occur concurrently. Additionally or alternatively, wherein the control circuitry is configured so that the first electrode is selected such that the target and the portion are not spatially overlapping, and the electrically modulating and optically modulating steps occur in an interleaving or alternating fashion.

Additionally or alternatively, the lead comprises a plurality of optical devices located at a first longitudinal position along the lead such that each optical device provides a spatially selective optical output. Additionally or alternatively, the lead comprises a plurality of segmented electrodes distributed in a set at a second longitudinal position along the lead, the set allowing a directional electrical signal to be defined.

Another illustrative and non-limiting example takes the form of a lead adapted for implantation in a patient, comprising: a proximal end configured for coupling to an implantable medical device pulse generator; a distal end comprising a plurality of optical elements and a plurality of electrodes; the plurality of optical elements spaced about a circumference of the distal end; and a shaft extending between the proximal end and the distal end and having therein a plurality of signal carriers for carrying separate signals from the pulse generator to the plurality of optical elements, allowing each optical element to be separately activated in a directionally specific manner.

Additionally or alternatively, the signal carriers are optical fibers, and the optical elements couple to the signal carriers to allow an optical signal from the optical fiber to be transmitted to patient tissue therethrough. Additionally or alternatively, the signal carriers are electrical connectors, and the optical elements are light emitters adapted to convert an electrical signal to an optical output to be transmitted to patient tissue.

Another illustrative and non-limiting example takes the form of an implantable medical device adapted for placement in the sphenoid sinus cavity of a patient, the medical device including operational circuitry and a power source, and having thereon one or more optical elements adapted to issue photobiomodulation (PBM) to patient tissue via the sphenoid sinus cavity, the operational circuitry adapted to use power from the power source to activate the one or more optical elements and issue PBM to the patient via the spenoid sinus cavity.

Additionally or alternatively, the PBM has a wavelength in the range of about 600 to 1000 nanometers.

Another illustrative and non-limiting example takes the form of a method of treating a patient comprising: optically modulating a target of at least one subsector or anatomic region of the basal forebrain by photobiomodulation (PBM) generated from a first optical device located at, adjacent to, or near the basal forebrain; and electrically modulating a portion of the basal forebrain with an electrical stimulus generated from a first electrode positioned on a structure that also carries the optical device.

Additionally or alternatively, the combination of optical modulating and electrical modulating prevents the degeneration of a patient's Nucleus Basalis of Meynert. Additionally or alternatively, the target is selected for the subsector or region of patient's basal forebrain in need of functional improvement.

Additionally or alternatively, the target and the portion are spatially overlapping, and the electrically modulating and optically modulating steps occur concurrently.

Additionally or alternatively, the target and the portion are spatially overlapping, and the electrically modulating and optically modulating steps occur in an interleaving or alternating fashion. Additionally or alternatively, the interleaving or alternating fashion includes a first period in which the target is modulated, and a second period in which the portion is modulated, and the first period overlaps a portion of the second period. Additionally or alternatively, the interleaving fashion includes a first period in which the target is modulated, and a second period in which the portion is modulated, and the first period does not overlap a portion of the second period.

Additionally or alternatively, the target and the portion are not spatially overlapping, and the electrically modulating and optically modulating steps occur concurrently. Additionally or alternatively, the target and the portion are not spatially overlapping, and the electrically modulating and optically modulating steps occur in an interleaving or alternating fashion. Additionally or alternatively, the interleaving fashion includes a first period in which the target is modulated, and a second period in which the portion is modulated, and the first period overlaps a portion of the second period. Additionally or alternatively, the interleaving fashion includes a first period in which the target is modulated, and a second period in which the portion is modulated, and the first period does not overlap a portion of the second period.

Additionally or alternatively, the device is a lead having proximal and distal ends, the proximal end configured for attachment to an implantable pulse generator and the distal end comprising the electrode and a plurality of optical devices located at a first longitudinal position along the lead such that each optical device provides a spatially selective optical output. Additionally or alternatively, the lead comprises a plurality of segmented electrodes distributed in a set at a second longitudinal position along the lead, the set allowing a directional electrical signal to be defined, and the first electrode and the first optical device are spatially aligned with one another along the lead. Additionally or alternatively, the optical device on the lead is configured for intracranial implantation in the brain. Additionally or alternatively, the optical modulation comprises optical energy having a wavelength in the range of about 600 to 1000 nanometers. Additionally or alternatively, the electrical signal is generated at a frequency in the range of about 20 to 80 Hz, for about 5 to about 30 seconds, followed by about 30 to about 55 seconds of rest.

Additionally or alternatively, the optical signal is generated in response to a determination that the patient has awoken from a period of sleep. Additionally or alternatively, the optical signal is issued to cause an increased blood flow and improved neural network activity in the region or the subsector in need of functional improvement. Additionally or alternatively, the optical PBM is provided prior to executing tasks to condition the functional area to enhance execution of a function or task associated with the region or the subsector in need of functional improvement.

Another illustrative and non-limiting example takes the form of a method of treating a patient comprising: optically modulating a target of at least one subsector or anatomic region of the basal forebrain by photobiomodulation (PBM) generated from a first optical device located at, adjacent to, or near the basal forebrain wherein the first optical device is placed in a sinus cavity in the patient's head; and electrically modulating a portion of the basal forebrain with an electrical signal generated from a first electrode wherein the first electrode is positioned on a lead that passes from a pulse generator through a bore hole in the patient's cranium and into the patient's brain.

Additionally or alternatively, the optical device is configured for removable placement in the sphenoid sinus cavity of the patient. Additionally or alternatively, the optical device is placed in or in proximity with the sphenoid sinus cavity. Additionally or alternatively, the combination of optical and electrical modulation is selected to prevent degeneration of a patient's Nucleus Basalis of Meynert. Additionally or alternatively, the optical device is configured to emit light wavelengths in the range of about 600 to 1000 nanometers. Additionally or alternatively, the electrical signal is generated at a frequency of in the range of about 20 to 80 Hz, for about 5 to about 30 seconds, followed by about 30 to about 55 seconds of rest.

Another illustrative and non-limiting example takes the form of an implantable lead comprising: a lead body extending between a proximal end and a distal end, the lead body having a plurality of electrical conductors passing therethrough; the proximal end including a connector for connecting to a pulse generator, the connector providing electrical connections between the plurality of electrical conductors and circuitry inside the pulse generator; a plurality of electrodes positioned on the lead body near the distal end, the plurality of electrodes connected to the plurality of electrical conductors; and a least first and second optical devices positioned on the lead body near the distal end, the first and second optical devices configured to output optical energy in first and second directions, respectively.

Additionally or alternatively, the lead includes a marker disposed on the lead body near the distal end, the marker providing a reference to allow determination of directions in which the first and second optical devices are pointed. Additionally or alternatively, the first and second optical devices are optical transducers, the optical transducers connected to the plurality of electrical conductors such that each optical transducer can be separately activated. Additionally or alternatively, the lead body includes at least first and second optical fibers coupled, respectively, to the first and second optical devices. Additionally or alternatively, the first and second optical devices are positioned at a single longitudinal position along the lead body.

This overview is intended to introduce the subject matter of the present patent application. It is not intended to provide an exclusive or exhaustive explanation. The detailed description below provides further details.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative DBS system implanted in a patient;

FIG. 2 illustrates details of a directional DBS lead;

FIG. 3 illustrates the details of an optrode lead;

FIG. 4 shows the intracranial placement of the optrode lead near the basal forebrain;

FIG. 5 illustrates placement of an optically emitting device in a nasal sinus of a patient;

FIG. 6 shows a device configured with optical output devices; and

FIG. 7 is a block diagram for illustrative methods of treatment.

### DETAILED DESCRIPTION

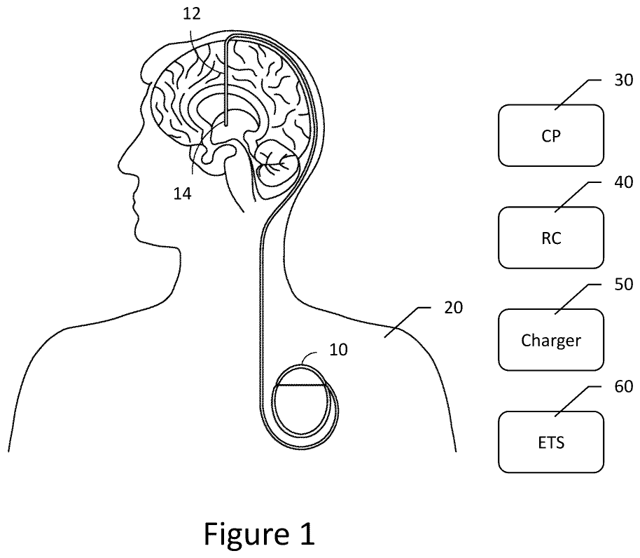

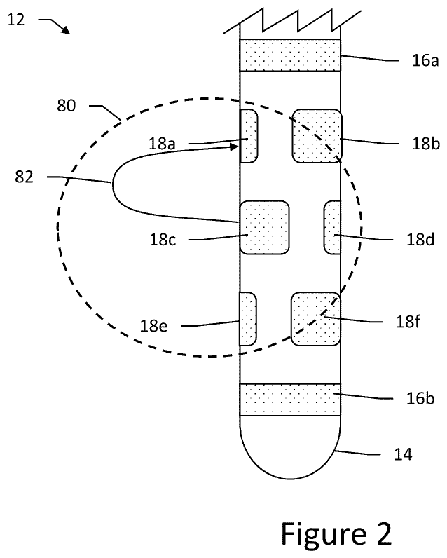

FIG. 1 shows an illustrative DBS system implanted in a patient. The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 can be used by a physician, or at the direction of a physician, to obtain data from and provide instructions the pulse generator 10 via suitable communications protocols such as Bluetooth or MedRadio or other wireless communications standards, and/or via other modalities such as inductive telemetry.

A patient remote control (RC) 40 can be used by the patient to perform various actions relative to the pulse generator 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 40 can communicate via similar telemetry as the CP 30 to control and/or obtain data from the pulse generator 10. The patient RC 40 may also be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator battery, using known methods and circuitry.

Some systems may include an external test stimulator (ETS) 60. The ETS 60 can be used to test therapy programs after the lead 12 has been implanted in the patient to determine whether therapy will or can work for the patient 20. For example, an initial implantation of the lead 12 can take place using, for example, a stereotactic guidance system, with the pulse generator 10 temporarily left out. The lead 12 may have a proximal end thereof connected to an intermediate connector (sometimes called an operating room cable) that couples to the ETS 60. After lead 12 has been implanted and coupled to the ETS 60, the ETS can be programmed using the CP 30 with various therapy programs and stimulation parameters. The patient 20 may then test the therapy programs using the RC 40 to select programs to test during a trial period. Once therapy suitability for the patient is established to the satisfaction of the patient 20 and/or physician, the permanent pulse generator 10 is implanted and the lead 12 is connected thereto, with the ETS then being removed from use.

The pulse generator 10 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and/or DBS. Additional background and/or examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244, 150, 7,672,734, 7,761,165, 7,974,706, 8,175,710, 8,224,450, and 8,364,278, the disclosures of which are incorporated herein by reference in their entireties.

FIG. 2 illustrates details of a directional DBS lead. The distal end 14 is shown, and a plurality of electrodes are shown as well. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 may be separately addressable in the system, such as by using a pulse generator having multiple independent current control (MICC) or multiple voltage sources.

MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes 16, 18 on the lead. It should be noted that the pulse generator canister may serve as an indifferent electrode or as a return electrode for therapy outputs. Alternatively, one of the lead electrodes (such as a ring electrode 16 or one or more of the segmented electrodes 18) may instead be used as a return electrode. Thus, for example, the electrodes 16, 18 on the lead may serve as cathodes while pulse generator canister serves as an anode during one phase of stimulation pulse delivery. In another example, some of the lead electrodes 16, 18 serve as cathodes, while other lead electrodes 16, 18 serve as anodes during one phase of stimulation pulse delivery.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

A directional lead as shown in FIG. 2 may be used to generate a stimulation field as illustrated at 80 in FIG. 2. The outer boundary of field 80 may be understood as representing an equipotential or equal field boundary, within which the electrical field is higher than an activation threshold, and outside of which the electrical field is below the threshold, for purposes of illustration. An activation threshold may represent or approximate a voltage/field strength at which neural cells will activate; activation thresholds may be determined on a population basis, such as by relating to a voltage/field at which a 50% likelihood of activation of 50% of the cell population is determined; other boundaries/thresholds can be used. The shape of the field can be adjusted, as described variously in the references incorporated by reference above, by modifying the fractionalization of current issued via the electrodes. An electrical field as shown at 80 may be (roughly) generated by using electrode 18c as a cathode, and surrounding electrodes 18a, 18e, and 18d as anodes, for example. The actual characteristics of fractionalization may be more sophisticated than this simple example.

A related concept to the field shown at 80 in FIG. 2 is that of stimulation field modeling (SFM). In SFM, the tissue is modeled, for example, using finite element models in which the lead body is treated as an insulator, surrounded by a thin encapsulation sheath, and surrounded by neural tissue. The neural tissue may be modeled as isotropic and homogenous, though more sophisticated modelling can also be used if desired. A set of model volumes are defined around the lead, breaking up the space into small blocks, each of which can be analyzed within the model. The outer boundaries of the SFM can be determined using a population-based activation threshold as described above. The result can be that at a given stimulation current, an SFM can be generated as a three-dimensional surface surrounding a portion of the lead and encompassing a volume of neural tissue. Field 80 may be, for example, understood as a two-dimensional representation of a slice of the SFM.

The positioning of the SFM relative to target and/or non-target (side-effect) neural structures may be determined for a given system using imaging modalities such as X-ray, CT scan, PET scan, MRI, f-MRI, etc., to identify lead position in the patient. Imaging system data for the patient may also be used to estimate the locations of neural structures in the patient including relative to the identified lead position. The imaging data may be merged or overlapped with general anatomical knowledge, such as from a brain atlas, which can help identify the particular structures that can be identified from imaging data. The use of SFM in association with a directional lead can allow therapy planning to precisely stimulate targeted tissue structures while avoiding excess stimulation of non-target tissue and limiting stimulation of structures associated with adverse side effects.

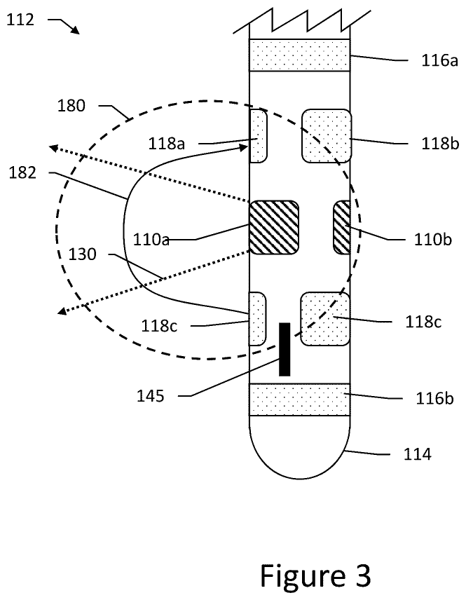

FIG. 3 illustrates details of an optrode lead 112 which comprises of a plurality of electrodes 116a, 116b (collectively 116), 118a, 118b, 118c, 118d (collectively, 118) and optical devices 110a, 110b (collectively optical devices 110). The electrodes are used to issue current/voltage to electrically stimulate 180 the target neural structure. As used herein, an “optrode lead” 112 includes each of optical devices 110 and electrodes 118. The optical devices 110 produce a directional optical output 130 which in turn causes PBM of the target. A radiopaque marker 145, which can be detected via X-ray to indicate position and direction of the electrodes 116, 118 and optical devices 110 relative to tissue, may be provided as well on the optrode lead 112.

PBM has been applied to promote healing and regeneration of various tissues and has been used in multiple fields including sports medicine, orthopedics, dentistry, diabetes, and dermatology. Therapeutic benefits may include, for example, and without limitation, improved tissue repair and regeneration, reduced inflammation, improved management of acute and chronic pain, enhancing mitochondrial function, reducing oxidative stress of the target, and enhancement of circulation and vasodilation. Such benefits can be produced with minimal side effects and damage to the target.

As it pertains to the neuroprotective effects of PBM, an increase in vasodilation may improve the circulation of blood to the target. Such enhancement promotes tissue healing and repair, improve oxygen and nutrient delivery to the target. Additionally, PBM may enhance mitochondrial activity and increase ATP production, directly affecting neural cells themselves in addition to providing the secondary effect of increased circulation. By promoting ATP production, PBM can help support the energy needs of and neurotransmission by targeted neurons and maintain cellular metabolism and homeostasis of ion pumps. By promoting ATP production, PBM can contribute to the health, survival, and functionality of the target neural tissue.

The effectiveness of PBM therapy to the particular target may be influenced by the choice of wavelength of light 130 emitted by the optical devices 110. Optical devices 110 may use a light source to generate a full field optical output around the lead, or to generate a spatially selective directional optical output 130 giving rise to PBM of the relevant target. The light source may be placed locally at the distal end of the lead or distantly at the proximal end of the lead. The light source may be, for example, a laser diode or a light-emitting diode (LED). Light delivery systems, such as optical fibers, can be employed to deliver the light (whether collimated or not) in such a way to direct the optical output 130 to the target with precision. Some examples may use an array of vertical cavity surface emitting lasers, for example. Some examples, rather than collimating light for a highly directional output, may use a convex surface to disperse light, if desired.

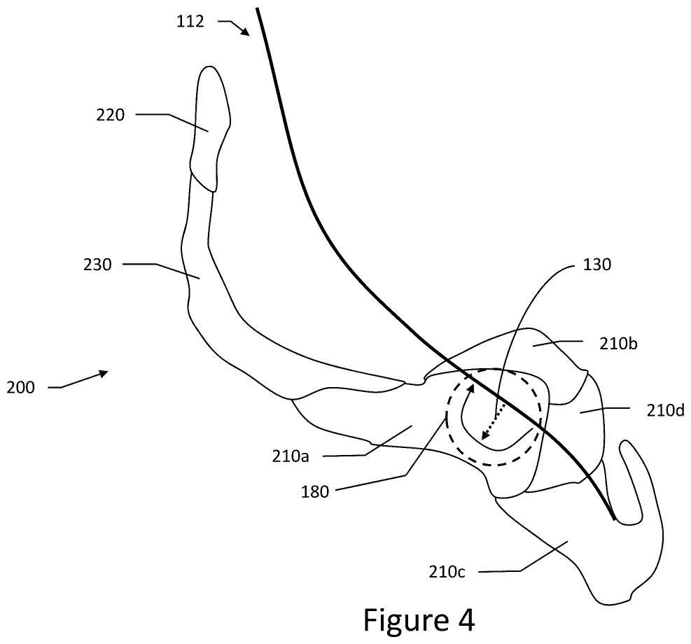

As shown further in the examples of FIGS. 3-4, the light source is placed near or in contact with the targeted tissue 210, thus allowing the optical output 130 to penetrate the target and induce the physiological reactions that generate the therapeutic benefits of PBM. Red light or near-infrared light of 600-110 nm may be used for inducing the desired therapeutic benefits of PBM. Other stimulation parameters that may influence the effect of PBM include, but is not limited to, the energy intensity of the light source, the power intensity and/or power density of the optical device 110, whether a continuous wave or pulsed light is issued, and the duration of the therapy. A plurality of selective wavelength sources may be used simultaneously, sequentially, singularly, or in other combinations or patterns, as desired.

The plurality of electrodes 116, 118 and optical devices 110 in FIG. 3 are arranged around the circumference of the lead 112, allowing the lead 112 to stimulate any target near the lead 112 without having to re-orient the lead 112. In use, the active optical devices 110 and electrodes 118 may be spatially aligned along the same longitudinal position of the lead 100, or, in the alternative, may be offset from one another. When aligned along lead 100, the electrodes 118 and the optical devices 110 are positioned in such a way to stimulate the same target. A marker 145 may be placed on the lead 112 to indicate the position and alignment of the lead 112 needed for precise and accurate optical and electrical stimulation 130, 182 of the target.

The optical devices 110 may be separately addressable so as to emit specific spatially selective directional optical outputs 130 suited to stimulate the chosen target. For example, optical devices 110 may emit a pre-determined, uniform optical output 130 or may be used with a compatible PBM control system to separately address the optical devices 110. PBM control systems are configured to adjust the stimulation parameters of PBM therapy best suited to bring about the desired therapeutic benefits to the target. Stimulation parameters that may be controlled by the PBM control system include, but are not limited to, selection of the wavelength of the optical output 130, the intensity and dosage of the optical output, the pulsing setting (PW, rate, and duty cycle etc) of the output, the duration and timing of optical outputs, and the schedule of PBM therapy. Control systems may include computer-controlled systems or programmable optical devices 110 to control the optical output 130.

In some examples, each light source 110 on the lead may be a transducer, such as a laser or diode or other light emitting device, each (separately) coupled electrically to the pulse generator by electrical connections through the body of lead 112. A common ground wire may be used by plural light transducers, as desired. Alternatively, one or more light sources may be provided in a pulse generator (not shown in FIG. 3), and coupled by a light guide, such as an optical fiber, through the lead.

Another design may be understood by reference to FIG. 3, with somewhat different understanding of certain parts. The tip 114 may be an optical element in addition to or in place of the light sources 110. For example, the tip 114 may include a dome shaped optically transparent material, acting as a dispersive lens that can provide an optical output. The dome shape and/or lens-like capability is optional; any suitable design to allow an optical output to be delivered at the tip can be used, if desired.

FIG. 4 illustrates the placement of an optrode lead 112 at or near the basal forebrain 200. More particularly, in FIG. 4, the lead 112 is placed near the NBM regions 210a, 210b, 210c, 210d (collectively 210) of the basal forebrain, which includes the anterior-medial (Ch4am) 210a, anterior-lateral (Ch4al) 210b, posterior (Ch4p) 210c, and intermediate (Ch4i) 210d NBM subregions 210. In this example, FIG. 4 shows the optical stimulation 130 and electrical stimulation 180 of the targeted anterior-medial subregion 210a at overlapping times. Selection of the target is not limited to the various subregions of the NBM, other regions of the basal forebrain containing cholinergic neurons are also eligible, including the medial septum (MS) 220 and the diagonal band of Broca (DB) 230. The selection of the region or subregion target is dependent on a variety of factors, including, but limited to, the desired therapeutic effect and the neural structure of the patient.

In some examples, a tissue region/volume that is stimulated by the electrical stimulation 180 may overlap at least a portion of a tissue region/volume that is also stimulated by the optical output, to increase therapeutic effect or to overlap two different mechanisms of therapy effects. Stimulation may be simultaneous both spatially and temporally, if desired. In other examples, electrical stimulation and optical stimulation may be interleaved, such as by temporally offsetting one relative to the other.

In some examples, optical stimulation of a region may be coordinated with electrical therapy designed to reduce signal transmission in neural tissue around the region of optical stimulation. This may reduce side effects of the optical stimulation from being transmitted to other parts of the body, if desired.

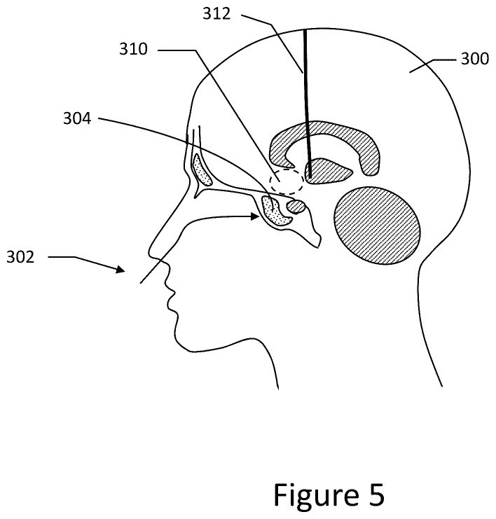

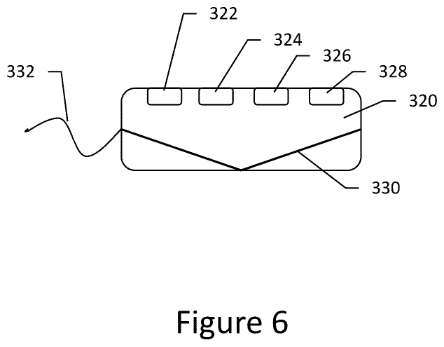

FIG. 5 shows a path for placement of a device in a sinus of a patient 300. Here, an intranasal path for placement of a device is illustrated at 302, extending to the sphenoid sinus 304. As an alternative to intracranial implantation of an optrode lead as illustrated in FIG. 3, a standard DBS lead 312 maybe positioned as shown, with optical stimulation provided from a different location in a less invasive manner. For example, a device as shown in FIG. 6, below, may be positioned in a sinus cavity of a patient. The use of the sphenoid sinus 304 is illustrative, and may be beneficial for its proximity to the basal forebrain and NBM, allowing region 310 to be stimulated with optical energy.

A device as shown in FIG. 6 may be placed. For example, the device 320 may include a plurality of optical output devices 322, 324, 326, 328, such as light emitting diodes, lasers, etc. As few as a single optical output device may be provided, or as many as are desired; four are shown for illustration. For optical stimulation of the NBM, it would be desirable to direct the optical devices toward the forebrain when emplaced.

Placed in contact with the nasal mucosa, optical stimulation may be administered through the nasal cavity via the device 320. The nasal mucosa has a high degree of vascularity and high amount of blood vessels. These features of the nasal mucosa allow the delivery of an optical output sufficient to generate PBM. The lead, as shown in FIG. 5, may be a standard DBS lead if desired. In an alternative, the lead may include an optical sensor configured to determine whether output optical energy from a device 320 is reaching the target tissue, allowing calibration of power level, wavelength, and/or spatial selection of optical sources. For example, FIG. 3 may be understood as alternatively showing optical sensors at 110a, 110b, facilitating detection that would confirm the optical outputs from device 320 provide desired levels of power/energy, etc. The intracranial lead can also be used to issue electrical therapy signals.

Because intranasal delivery of the optical 130 stimulation to the basal forebrain is indirect in its nature, considerations in determining the stimulation parameters may not be consistent with the stimulation parameters applied to intracranial leads. Adjusting the stimulation parameters may be necessary to limit stimulation of avoid regions due to the limited precision and localization of intranasal stimulation and the differing anatomy and nasal conditions of the patient 20.

In FIG. 5, the device can be inserted through the nose or throat, and placed in or in proximity to the sphenoid sinus 304. The sphenoid sinus 304 may be selected because it is both accessible through intranasal placement and proximate to the basal forebrain. However, selection of the intranasal placement of the device 320 is not limited to the sphenoid sinus 304. Other paranasal sinuses (the frontal sinuses, axillary sinuses, and ethmoid sinuses) may be selected.

In still another example, the device 320 of FIG. 6 may include one or more markers 330, such as radiopaque markers that can allow a determination of the orientation of the device 320 once place. The device 320 may include a tether 332 that can be used for device retrieval; a hook or other structure to which a retrieval device can be attached may be included instead or in addition to the tether 332. Device 320 may be internally powered, such as by a battery if desired. Alternatively, the device 320 may be externally powered, such as by including an inductive transducer to receive power from a varying magnetic field applied to the patient's cranium.

In another example, the device 320 may be used in conjunction with an optrode as shown in FIG. 3 for purposes of configuring device parameters. For example, device 320 may have one or more optical receivers at any of 322, 324, 326, 328, and may be placed intracranially in a sinus, such as the sphenoid sinus 304. The optrode may then be activated and optical energy issued while the optical receiver on device 320 is used to determine whether light energy is being transmitted to the correct tissue at desired amplitudes. Following confirmation that therapy is reaching targeted tissue, the device 320 may be removed, if desired.

In another alternative, optical and/or electrical sensors may be positioned on a catheter, a guidewire, or other intravascular device that can be threaded through the vasculature to a desired location near the basal forebrain and/or NBM, preferably with the aid of fluoroscopic guidance. With the optical and/or electrical sensors positioned as desired, the optical and/or electrical therapy can be generated, and the sensors used to confirm that therapy is reaching target tissue in desired quantities/amplitudes/power levels.

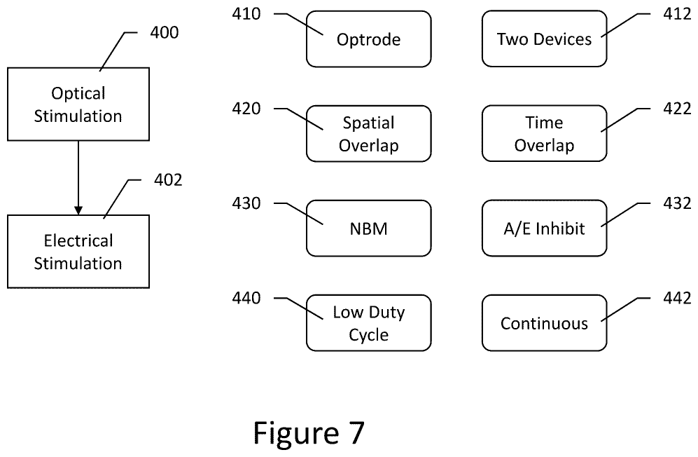

FIG. 7 shows illustrative methods in block form, with several options illustrated. Optical stimulation is delivered at step 400, and electrical stimulation is delivered at step 402. The paired therapy can be delivered from a single unit or optrode, as indicated at 410. Alternative, paired therapy can be delivered using two devices, as indicated at 412, such as by use of a lead implanted in the brain and an optical output device placed (temporarily or permanently) in the sphenoid sinus of the patient, or elsewhere, preferably in the nasal sinuses. In yet another alternative, paired therapy may be delivered using an optrode at 410 for both electrical and optical stimulation, and a second (or more) device may be positioned to verify that stimulation reaches the desired target tissue, using for example a device placed in a nasal sinus and/or in a blood vessel.

The optical stimulation 400 and electrical stimulation 402 may be applied to the same volume of tissue, if desired, with spatial overlap 420, as well as at the same point in time, with time overlap 422. Spatial overlap 420 may instead be avoided, if desired, so that optical stimulation applies to one region of tissue, and electrical stimulation to a separate region of tissue, if desired. Time overlap 422 may be used, or may be avoided, to different ends. In one example, electrical stimulation may be used to inhibit passing of afferent or efferent signals, as indicated at 432, limiting side effects of optical stimulation from manifesting.

For example, optical stimulation, to the extent it increases vasodilation, may be used to improve regional blood flow and maintain viability of tissue in a given volume, and to prevent such stimulation from interfering with sleep patterns, electrical stimulation may be used to inhibit signaling. For this example, spatial overlap 420 may not be used, but time overlap 422 would be used, and the goal of the electrical therapy 432 would be to inhibit resultant signaling 432. Other combinations can be used; this example is prophetic. The stimulation, whether optical or electrical, may be directed to the NBM, as indicated at 430, and/or any of the structures highlighted above in FIG. 4.

Different amounts of therapy can be used. For example, a very low duty cycle 440 may be used for issuing therapy directed to long term effects, for example, preserving tissue viability, while continuous therapy 442 may be used to provide immediate symptom relief. In an example, an optrode is used to issue electrical therapy to the NBM to provide alleviation of dyskinesias for a patient having Parkinson's disease. Optical therapy may also be delivered, but at a lower duty cycle or in a manner that is independent of alleviation of the dyskinesia. For example, optical stimulation 400 could be delivered for one minute of every hour throughout the day (duty cycle of about 2%), while electrical stimulation 402 is delivered on a more continuous basis during waking hours to aid in muscle control.

In some examples optical therapy may be generated in a more or less continuous manner, while electrical stimulation may be used more sporadically or at lesser duty cycle. For example, some therapy combinations have been shown to have long lasting effects if delivered for minutes, up to one to two hours per day, whether in a single shot or spread out during the day. Alternatively, optical therapy may be delivered for a short period of time each day, either on a scheduled basis or in response to an input or event. For example, optical therapy could be delivered for a period of seconds or minutes each time the system determines that the patient has awakened from a period of sleep (whether overnight or a nap), while electrical therapy could be issued on demand or on a continuous basis. A determination that the patient has awakened may be made using, for example, a motion sensor or accelerometer that is either integral to the system (such as in the pulse generator), or which may be worn by the patient but allowed to communicate with the patient RC or implanted system, for example. In other examples, the patient may indicate a request for one or the other therapy, as desired.

Time overlap 422 may include interleaving two stimulation types in a multi-modal therapy. For example, optical stimulation 400 may be performed as a pulse train for a time period, with a quiescent period separating pulse trains. The electrical stimulation 402 may be performed during quiescent periods of the optical stimulation. More particularly, for example, optical stimulation may be performed for a period of 10 milliseconds to 60seconds, with gaps of 10 milliseconds to 60 seconds of quiescent period, during which electrical stimulation 402 is performed. Interleaving stimulation does not need to be symmetric. For example, optical stimulation may include issuing 20 to 40 light pulses of 1 millisecond duration with 4 milliseconds therebetween as an optical pulse train of 100 to 200 millisecond duration, and electrical stimulation may include issuing 20 to 40 electrical pulses of 100 microseconds duration with 200 microseconds therebetween, for an electrical pulse train of 6 to 20 milliseconds duration. Once the electrical stimulation is completed, the optical stimulation again starts in this example.

Electrical stimulus may be delivered for a period of, for example, about 5 to about 30 seconds, with rest periods of about 30 to about 55 seconds, in some examples. In one example, electrical stimulus is conducted at a frequency of 20 to 80 Hz for 20 seconds, followed by 40 seconds of rest. Optionally, the optical stimulation 400 may be issued during a portion of the 40 seconds of rest. Such stimulus may be performed at a desired time of day for the patient, such as late morning, or approximately 2-4 hours after the patient awakens from overnight rest.

In some examples, electrical signal delivery may have a non-stimulating effect, such as to down-regulate one or more neural networks or activities in the tissue subject to the electrical pulses. Thus, some examples herein may refer to electrical modulation, which means the issuance of electrical signals that may include both electrical signals having stimulatory effects as well as down-regulating effects. The same is true for optical stimulation. Optical outputs may cause stimulation and/or down-regulation of neural activity. Optical modulation encompasses optical outputs that cause stimulation as well as optical outputs that cause down-regulation of neural activity.

While much of the above discussion focuses on use for brain tissue, other tissue regions can also be treated. These may include structures to be treated in spinal cord stimulation (SCS), peripheral nerve stimulation, occipital nerve stimulation, muscles and muscle nerve fibers, therapies directed to the digestive tract or other regions, and vagus nerve stimulation. Combination therapies using both electrical and optical stimulation can be paired for other uses. In addition, lead designs including, for example, directional or segmented electrode designs as well as directional optical outputs, may be used in other locations. Of particular note is that a design with multiple optical output elements arranged around the circumference of the lead may allow selective optical stimulation to be configured for a given patient and/or neural structure.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient comprising:
optically modulating a target of at least one subsector or anatomic region of the basal forebrain by photobiomodulation (PBM) generated from a first optical device located at, adjacent to, or near the basal forebrain; and
electrically modulating a portion of the basal forebrain with an electrical stimulus generated from a first electrode positioned on a structure that also carries the optical device.

2. The method of claim 1, wherein the combination of optical modulating and electrical modulating prevents the degeneration of a patient's Nucleus Basalis of Meynert.

3. The method of claim 1, wherein the target is selected for the subsector or region of patient's basal forebrain in need of functional improvement.

4. The method of claim 1, wherein the device is a lead having proximal and distal ends, the proximal end configured for attachment to an implantable pulse generator and the distal end comprising the electrode and a plurality of optical devices located at a first longitudinal position along the lead such that each optical device provides a spatially selective optical output.

5. The method of claim 4, wherein the lead comprises a plurality of segmented electrodes distributed in a set at a second longitudinal position along the lead, the set allowing a directional electrical signal to be defined, and the first electrode and the first optical device are spatially aligned with one another along the lead.

6. The method of claim 1, wherein the optical modulation comprises optical energy having a wavelength in the range of about 600 to 1000 nanometers.

7. The method of claim 1, wherein the electrical signal is generated at a frequency in the range of about 20 to 80 Hz, for about 5 to about 30 seconds, followed by about 30 to about 55 seconds of rest.

8. The method of claim 1, wherein the optical signal is generated in response to a determination that the patient has awoken from a period of sleep.

9. The method of claim 1, wherein the optical PBM is issued to cause an increased blood flow and improved neural network activity in the region or the subsector in need of functional improvement.

10. The method of claim 9, wherein the optical PBM is provided prior to executing tasks to condition the functional area to enhance execution of a function or task associated with the region or the subsector in need of functional improvement.

11. A method of treating a patient comprising:
optically modulating a target of at least one subsector or anatomic region of the basal forebrain by photobiomodulation (PBM) generated from a first optical device located at, adjacent to, or near the basal forebrain wherein the first optical device is placed in a sinus cavity in the patient's head; and
electrically modulating a portion of the basal forebrain with an electrical signal generated from a first electrode wherein the first electrode is positioned on a lead that passes from a pulse generator through a bore hole in the patient's cranium and into the patient's brain.

12. The method of claim 11, wherein the optical device is configured for removable placement in the sphenoid sinus cavity of the patient.

13. The method of claim 11, wherein the first optical device is placed in or in proximity with the sphenoid sinus cavity.

14. The method of claim 11, wherein the combination of optical and electrical modulation is selected to prevent degeneration of a patient's Nucleus Basalis of Meynert.

15. The method of claim 11, wherein the optical device is configured to emit light wavelengths in the range of about 600 to 1000 nanometers.

16. The method of claim 11, wherein the electrical signal is generated at a frequency of in the range of about 20 to 80 Hz, for about 5 to about 30 seconds, followed by about 30 to about 55 seconds of rest.

17. An implantable lead comprising:
a lead body extending between a proximal end and a distal end, the lead body having a plurality of electrical conductors passing therethrough;
the proximal end including a connector for connecting to a pulse generator, the connector providing electrical connections between the plurality of electrical conductors and circuitry inside the pulse generator;
a plurality of electrodes positioned on the lead body near the distal end, the plurality of electrodes connected to the plurality of electrical conductors; and
a least first and second optical devices positioned on the lead body near the distal end, the first and second optical devices configured to output optical energy in first and second directions, respectively.

18. The lead of claim 17, further comprising a marker disposed on the lead body near the distal end, the marker providing a reference to allow determination of directions in which the first and second optical devices are pointed.

19. The lead of claim 17, wherein the first and second optical devices are optical transducers, the optical transducers connected to the plurality of electrical conductors such that each optical transducer can be separately activated.

20. The lead of claim 17, wherein the lead body includes at least first and second optical fibers coupled, respectively, to the first and second optical devices.
